(19) United States
(12) Patent Application Publication
(10) Pub. No.: US 2010/0291591 A1
(43) Pub. Date: Nov. 18, 2010

(54) NOVEL PLATING MEDIA

(75) Inventors: Lukas Wick, Winterthur (CH); Guenter Schabert, Goldach (CH); Urs Spitz, Herriberg (CH)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carisbad, CA (US)

(21) Appl. No.: 12/740,929

(22) PCT Filed: Nov. 4, 2008
(86) PCT No.: PCT/US08/82358

§ 371 Date: Jul. 29, 2010

Related U.S. Application Data
(60) Provisional application No. 60/985,615, filed on Nov. 5, 2007.

(51) Int. Cl. G01N 33/573

(52) U.S. Cl. 435/7.4

(57) ABSTRACT

Plating media contains enzyme substrates (ES) used to detect enzyme activity associated with a target organism and that include a binding motif (BM) that serves to bind detectable enzymes (DE) (also called marker enzymes in this disclosure). A signalogenic substructure (SG) produces detectable signals and an enzyme labile group (ELG) is labile to the action of a marker enzyme and it typically includes the binding motif. The enzyme labile group links to the signalogenic substructure via a labile bond (LB) which is cleaved by the action of the marker enzyme separating the signalogenic substructure and the enzyme labile group. In some embodiments a linker is inserted between the signalogenic substructure and the enzyme labile group. Presence of the enzyme and thus the organism is detected by presence of a fluorogenic, precipitate that can be detected in single colonies.

### RELATED APPLICATIONS

This application claims the benefit of U.S. Provisional Application No. 60/985,615, filed Nov. 5, 2007, the disclosure of which is incorporated herein by reference in its entirety.

### FIELD OF THE INVENTION

This disclosure relates to incorporating fluorogenic substrates into plating media for detecting enzyme activity of microbial species. The enzyme acts on the appropriate substrate to yield fluorescent products that are specifically formed, non-toxic, and insoluble in aqueous systems.

### BACKGROUND

Enzyme activity is useful in analyzing biological or chemical samples. It can indicate the presence of microbial organisms, including but not limited to archae, algae, bacteria, fungi, yeast, viruses and phages. Microbial organisms produce detectable marker enzymes that interact with enzyme substrates to yield detectable signals. Various assays are employed to identify and label the detectable signals.

### Enzyme Substrate Labeling Systems in Plating Media

Fluorogenic Substrates

Enzyme substrates are usually chromogenic or fluorogenic, and they can be further classified by solubility of the signalophore. Many enzyme assays are solution based. Currently available fluorogenic substrates typically yield soluble signalogenic substrates which tend to diffuse rapidly. Restaino et al. describe the use of a soluble fluorogenic enzyme substrate in plating media. Restaino et al., J. Food Prot. 62, 244-51 (1999). Soluble signalophores can only indicate the possible presence of a certain type of microbial species, and enzyme activity cannot be localized to individual colonies. Therefore, there is a need for a fluorogenic substrate that produces insoluble signalophores that allow precise staining and localization of individual colonies.

Indoxyl Substrates

Indoxyl substrates, such as X-gal, are widely employed and used as chromogenic substrates in plating media and otherwise to visualize enzymatic activity in microbiology, immunology, biochemistry and genetics. A large selection of these substrates is available for virtually any hydrolytic enzyme. In course of the assay, the indoxyl chromogen is set free by enzymatic hydrolysis and undergoes oxidative dimerization to yield colloidal indigo dyes. Enzyme substrates that yield such insoluble dyes allow for physical localization of enzymatic activity.

Indoxyl substrates are often used in combinations for dual or multiple assays because a number of indoxyl chromogens are available that produce different colors of indigo dyes upon oxidative dimerization. Two or more enzymes can be simultaneously detected if contrast and shade of color are sufficiently different.

Chromogenic indoxyl substrates play a significant role in biological research, commercial diagnostics, testing, and standard molecular biology applications such as western blotting or reporter gene assays. Despite their widespread use and commercial significance, indoxyl substrates have several limitations. For example, indoxyl substrates depend on the presence of molecular oxygen or other suitable oxidizers to develop the desired signalophore (indigo stain). This is intrinsic to the structure of indigo, formed when two indoxyl moieties undergo oxidative dimerization. Due to this requirement, indoxyl substrates are of limited or no use under anaerobic conditions. Therefore, there is a need for enzyme substrates that can function in the absence of molecular oxygen.

Furthermore, the formation of indigo is believed to involve a radical chain reaction. Such mechanism was proposed by Cotson and Holts et al., Pro. R. Soc. Lond. B. Biol. Sci. 148, 506-19 (1958). This reaction mechanism involves a number of reactive free radical intermediates known to be highly toxic. This offers a plausible explanation for observations that bacterial growth is frequently inhibited if indoxyl substrates are used in an assay. For example, when Butterworth et al., studied the potential use of indoxyl ribopyranoside for diagnostic purposes, they observed significant levels of bacterial growth inhibition which was absent when other substrates were used. Butterworth et al., J. Appl. Microbiology 96, 170-76 (2004). Thus, there is a need for non-toxic enzyme substrates that do not inhibit microbial growth.

Metal Chelating Substrates

Another class of precipitating substrates is based on metal chelating molecules that form insoluble complexes with metal ions. Chelating molecules contain two or more functional groups that coordinate to a metal ion. These functional groups can be masked by an enzyme labile group to prevent the formation of a metal complex. An enzyme that removes the enzyme labile group can be detected in a system that contains the masked chelating molecule and a suitable concentration of a metal ion. The appearance of the corresponding metal complex can be indicative of the presence and activity of an enzyme. For example, substrates derived from 8-hydroxyquinoline such as 8-hydroxyquinoline-beta-D-glucuronide or the naturally occurring esculetin (esculin-beta-D-glucopyranoside) can be used to detect the corresponding enzymes in the presence of iron (III) salts.

Metal chelating substrates, however, have several limitations. Many of these chelating molecules are toxic. This toxicity is intrinsic to any metal chelating agent because they generally interfere with the functioning of metallo-enzymes. Second, the concentration of metal ions required for the assay often causes undesirable interference. Finally, most stains produced are brown or black and hence are not useful for dual enzyme assays. Thus, there is a need for enzyme substrates that are non-toxic, non-interfering, and capable of dual enzyme assays.

Diazonium Substrates

Diazonium stains are well known and often used to localize enzyme activity. This important method was pioneered by Seligman et al., J. Histochem. Cytochem., 2, 209-29 (1954); Burstone et al., J. Histochem. Cytochem., 4, 217-26 (1956); and Rutenburg et al., J. Histochem. Cytochem., 6, 122-29 (1958). It is based on the reaction of stabilized diazonium salts with electron rich aromatic amines and phenols to form azo dyes. Many azo dyes are intensely colored and some are fluorescent.

This diazonium coupling reaction will proceed with aromatic amines and phenols much faster than with their corresponding esters and amides. Therefore, hydrolytic enzyme activity can be detected by exposing a sample to a suitable phenolic ester or anilide followed by staining with diazonium salts. This assay results in good enzyme activity localization when the proper substrate and diazonium salts are selected. Numerous substrates and diazonium staining salts are commercially available. Pearson et al., Proc. Soc. Exptl. Biol. Med. 108, 619-13 (1961); Pearson et al., Lab Invest., 12, 712-20 (1963); Yarborough et al., J. Reticuloendothelial Soc., 4, 390-408 (1967); Grossau et al., Enzyme Histochemistry 397-404 (1987) and others developed staining methods that are based on the reaction of an indoxyl and 3-aminoindole chromogens with diazoinum electrophiles for use in histology. Mohler and Blau have evaluated many combinations of indoxyl beta-D-galactosidase substrates and commercial diazoinum salts. They reported that some azo dyes arising from such reactions display significant fluorescent properties. A combination of 5-bromo-6-chloro-3-indoyl-beta-D-galactopyranoside in combination with Fast Violet LB, referred to as Fluoro-X-Gal, proved useful in fluorescent staining of galactosidase activity in fixated cells. Mohler and Blau, Proc. Nat'l Acad. Sci. USA 12423-12427 (1996)

Diazonium substrates are limited, however, because diazonium salts are hazardous, carcinogenic, and highly toxic to humans and cell cultures. This method poses significant danger to the user and it is not compatible with in vivo or non-destructive types of assays. Hence, these substrates are not very useful in the context of microbial plating media. Therefore, there is a need for an enzyme substrate that would avoid the hazardous nature of diazonium salts, but retain the sold state fluorescence provided by some of these substrates for staining microbial colonies on plating media.

In summary, the prior art reveals a need for novel plating media incorporating enzyme substrates that satisfy the needs for (1) non-toxic substrates that do not inhibit microbial growth or endanger the user, (2) substrates that function in anaerobic environments, (3) insoluble substrates that display increased sensitivity for labeling individual colonies, (4) substrates that do not depend upon reagents that can interfere with the enzymatic functioning of the target microbial organism, and (5) substrates that provide better differentiation for dual enzyme assays.

### SUMMARY

Enzyme substrates (ES) of the disclosure used to detect enzyme activity typically consist of a combination of certain chemical substructures. FIG. 1 details three combinations of enzyme substrates. Some substructures are common to all types of disclosed enzyme substrates (shown in FIG. 1A). The binding motif (BM) serves to bind detectable enzymes (DE) (also called marker enzymes in this disclosure). The signalogenic substructure (SG) produces detectable signals. The enzyme labile group (ELG) is labile to the action of a marker enzyme and it typically includes the binding motif The enzyme labile group links to the signalogenic substructure via a labile bond (LB) which is cleaved by the action of the marker enzyme separating the signalogenic substructure and the enzyme labile group.

Some enzyme substrates contain a fourth substructure, the labile structure or spacer (LS). It is inserted between the signalogenic substructure and the enzyme labile group such that the labile bond is located between labile spacer and enzyme labile group as shown in FIG. 1B. The labile spacer separates the signalogenic substructure and enzyme labile group by two or more chemical bonds. It may be eliminated spontaneously following action of the marker enzyme on the enzyme substrate removing the enzyme labile group. The labile spacer may also be eliminated by other means, for example by an auxiliary chemical reagent or photo irradiation where the enzyme substrate is inert to such means prior to the action of the marker enzyme (FIG. 1B). The purpose of the labile spacer is to eliminate undesirable interaction between the signalogenic substructure and the enzyme labile group, for example, spontaneous hydrolysis that causes non-enzymatic cleavage of the labile bond that produces non-specific background signal. A labile spacer also offers expanded design options for enzyme substrates by enabling additional combinations of signalogenic substructures with enzyme labile groups. For example, a phenolic signalogenic substructure can be combined with oxygen linked enzyme labile groups such as glycosides and esters (LB: O—C) but not with nitrogen linked enzyme labile groups corresponding to amino acids or peptides (LB: N—C). Adding a labile spacer solves the problem by, for example, attaching to the oxygen of the signalogenic substructure and providing a nitrogen atom connecting to an amino acid or peptide enzyme labile group. See FIG. 1C for an example of this mechanism.

The enzyme substrate (with or without a labile spacer) is acted upon by a marker enzyme, resulting in removal of the enzyme labile group which can be seen as unmasking or unblocking the signalogenic substructure. The signalogenic substructure is activated by this process, thus forming what will be called an “active signalogen” (denoted as aSG in FIG. 1). Active signalogens generate “signalophores” (denoted as SP in FIG. 1) either spontaneously or by the effect of chemical agents, reagent enzymes, irradiation, electric fields or other means. Generation of signalophores from active signalogens often includes deprotonation, oxidation, hydrolysis, metal complexation or other chemical reactions. These reactions may involve chemical reagents as well as reagent enzymes. The active signalogen and the signalophore may be of different origin and chemically completely unrelated, in which case the active siganolgen acts as an agent needed to generate the signalophore from a reagent precursor.

Typically, detectable signal (denoted as DS in FIG. 1) associated with the signalophore is released during this process. It may be of transient or persisting nature and it may occur spontaneously, or it may require appropriate interrogation means such as optical excitation.

An embodiment of the present disclosure can be described as plating media comprising an enzyme substrate represented by the general structure “BLOCK-O—X” wherein O—X is a fluorogen, BLOCK is an enzyme group labile to the action of a marker enzyme, X and BLOCK are linked by 0, an oxygen atom providing a bond labile to the action of a marker enzyme that cleaves BLOCK from X—O, which aggregates to yield an insoluble fluorescent precipitate. The fluorogen substructure can include, but is not limited to, quinazolinones (quinazolones), benzimidazoles, benzothiazoles, benzoxazoles, quinolines, indulines, and phenathridines as described in U.S. Pat. No. 5,316,906 which is incorporated herein in its entirety by reference.

A further embodiment of the present disclosure can be described as a plating media comprising an enzyme substrate represented by the general structure “BLOCK-LS-O—X” wherein O—X is a fluorogen, BLOCK is an enzyme group labile to the action of a marker enzyme, O—X and BLOCK are linked by LS which represents a labile spacer providing a bond labile to the action of a marker enzyme resulting in the separation of BLOCK from LS-O—X, and where LS-O—X represents a moiety that upon loss of LS is converted to a second moiety that aggregates to yield an insoluble fluorescent precipitate.

A further embodiment of the present disclosure can be described as a method of detecting a microbial organism that expresses a marker enzyme comprising the steps of inoculating a plating media with a test sample, wherein the plating media comprises an enzyme substrate represented by the general structure “BLOCK-O—X” wherein O—X is a fluorogen, BLOCK is an enzyme group labile to the action of a marker enzyme, X and BLOCK are linked by 0, an oxygen atom providing a bond labile to the action of a marker enzyme that cleaves BLOCK from O—X, which aggregates to yield an insoluble fluorescent precipitate, incubating the plating media for a sufficient period to obtain colonies, and examining the plating media for fluorescent staining associated with an individual colony wherein the fluorescent staining is indicative of the presence of a microbial organism.

A further embodiment of the present disclosure can be described as a method of detecting a microbial organism that expresses a marker enzyme of the present invention comprising the steps of inoculating a plating media with a test sample, wherein the plating media comprises an enzyme substrate represented by the general structure “BLOCK-LS-O—X” wherein LS-O—X is a fluorogen, BLOCK is an enzyme group labile to the action of a marker enzyme, O—X and BLOCK are linked by LS which represents a labile spacer providing a bond labile to the action of a marker enzyme resulting in the separation of BLOCK from LS-O—X and where LS-O—X represents a moiety that upon loss of LS is converted to a second moiety that aggregates to yield an insoluble fluorescent precipitate, incubating the plating media for a sufficient period to obtain colonies, and examining the plating media for fluorescent staining associated with an individual colony wherein the fluorescent staining is indicative of the presence of a microbial organism.

In certain embodiments of the present disclosure, the X component of the fluorogen has the structure:

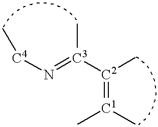

where carbon atoms of —C¹═C²— are further joined so as to complete a first 5- or 6-membered aromatic ring which may contain at least one of the hetero atoms N, O or S, carbon atoms of —C⁴—N═C³— are further joined so as to complete a second 5- or 6-membered aromatic ring that contains at least the nitrogen between C³ and C⁴ and may contain at least one additional hetero atom N, O or S, the first and second aromatic rings may be joined by a 5- or 6-membered bridging ring that contains at least the C² from the first aromatic ring and the C³ from the second aromatic ring, which bridging ring may be saturated or unsaturated and may contain a hetero atom N, O, or S, each of the first and second aromatic rings may be fused to at least one additional aromatic ring that may contain at least one of the hetero atoms N, O or S, and each of the aromatic rings may be further modified by substitution of any hydrogens on an aromatic carbon by substituents that are halogen, nitro, cyano, aryl, lower alkyl (1-4 carbons), perfluoroalkyl (1-4 carbons), or alkoxy (1-4 carbons), or any combination thereof; and X is covalently linked to the oxygen —O— at C¹.

In certain embodiments of the present disclosure, the marker enzyme, also referred to as the detectable enzyme, can be but is not limited to a glycosidase, peptidase, esterase, carboxylesterase, lipase, cholinesterase, phosphatase, sulfatase, phospholipase A, phospholipase B, phospholipase C, dealkylase or nitroreductase.

In certain embodiments of the present disclosure, the marker enzyme, also referred to as the detectable enzyme is a glycosidase. The glycosidase can be but is not limited to alpha-amylase, alpha-D-arabinosidase, alpha-L-arabinosidase, beta-D-cellobiosidase, alpha-D-fucosidase, alpha-L-fucosidase, beta-D-fucosidase, beta-L-fucosidase, alpha-gaLactosaminidase, beta-galactosaminidase, alpha-galactosidase, beta-galactosidase, alpha-glucosamimidas e, beta-glucosaminidase, alpha-glucosidase, beta-glucosidase, beta-glucuronidase, beta-lactosidase, alpha-maltosidase, beta-maltosidase, alpha-mannosidase, beta-mannosidase, neuraminidase, alpha-rhamnosidase, alpha-xylosidase, beta-xylosidase, alpha-L-arabinofuranosidase, beta-chitobiosidase, galactopyranoside-6-sulfatase and beta-D-ribofuranosidase.

In certain embodiments of the present disclosure, the marker enzyme, also referred to as the detectable enzyme is a peptidase. The peptidase can be but is not limited to L-alanine aminopeptidase, aminopeptidase A, aminopeptidase B, aminopeptidase M, dipeptidyl-aminopeptidase I, dipeptidyl-aminopeptidase II, dipeptidyl-aminopeptidase III, dipeptidyl-aminopeptidase IV, gamma-glutamyl transferase, hippurase, L-proline aminopeptidase, proline arylamidase, prolyl endopeptidase, proglutamyl peptidase and ureidase.

In certain embodiments of the present disclosure, the marker enzyme, also referred to as the detectable enzyme is a phosphatase, the phosphatase can be but is not limited to alkaline phosphatase, acidic phosphatase, phosphodiesterase, endo pyrophosphatase, exo pyrophosphatase, DNAse and ATPase.

In certain embodiments of the present disclosure, the marker enzyme, also referred to as the detectable enzyme is a sulfatase. The sulfatase can be but is not limited to arylsuflatase and glycosulfatase.

In certain embodiments of the present disclosure, the marker enzyme, also referred to as the detectable enzyme is a phospholipase C. The phospholipase C can be but is not limited to inositol specific phospholipase C and choline specific phospholipase C.

In certain embodiments of the present disclosure, the BLOCK group comprises a blocking group that is a monovalent moiety derived by removal of a hydroxyl group from phosphate or sulfate or a biologically compatible salt thereof; or a monovalent moiety derived by removal of a hydroxyl group from a carboxy group of an aliphatic, aromatic or amino acid or of a peptide; or a monovalent moiety derived by removal of an anometic hydroxyl group from a mono- or polysaccharide; and is capable of being cleaved from the remainder of the substrate. The phrase “capable of being cleaved” means that under physiological conditions, when the marker enzyme is present, it acts upon the BLOCK-O—X moiety to cleave BLOCK.

In certain embodiments of the present disclosure, the plating media can be in a dehydrated or powder form. As used herein, a dehydrated media contains less than about 10%, less than about 5%, less than about 1% or less than about 0.5% by weight of water. The powder form may be hydrated to yield a liquid or semi-liquid medium.

In certain embodiments of the present disclosure, X can be but is not limited to 6-chloro-2-(5-chloro-2-hydroxyphenyl)quinazolin-4(3H)-one (sold commercially by Molecular Probes, Inc. as ELF®).

A further embodiment of the present disclosure is a method for detecting methicillin resistant strains of S. aureus (MRSA). The detectable enzyme is S. aureus phosphatase, the enzyme substrate is 6-chloro-2-(5-chloro-2-hydroxyphenyl)-quinazolin-4(3H)-one phosphate, and the fluorescent staining associated with a colony is indicative of the presence of MRSA. This embodiment may also comprise the step of detecting potentially interfering colonies of Bacillus spp., Enterococci, or Serratia spp. by the addition of an indoxyl substrate, that can be but is not limited to 5-bromo-6-chloro-indolyl-beta-D-glucopyranoside. In the presence of Bacillus spp., Enterococci, or Serratia spp., the indoxyl substrates are hydrolysed and then undergo oxidative dimerization to yield indigo dyes that stain the interfering colonies purple.

A further embodiment of the present disclosure is a plating media for the detection of MRSA. In this embodiment, the plating media comprises 6-chloro-2-(5-chloro-2-hydroxyphenyl)-quinazolin-4(3H)-one phosphate sodium salt, 5-bromo-4-chloro-3-indoxyl-β-D-glucopyranoside, and additives for growth. The additives for growth include, but are not limited to, cefoxitine, sulbactam, polymyxin B, desferrioxamine B, ferrioxamin E, proteose peptone, bacto peptone, tryptone, yeast extract, meat extract, NaCl, LiCl, cycloheximid, agar, and water. This embodiment of the present disclosure can also be in powder form, containing less than 5% by weight of water. The powder form may be hydrated to yield a liquid or semi-liquid medium.

A further embodiment of the present disclosure is a plating media for the detection of Listeria monocytogenes, a common food borne pathogen. In this embodiment, the plating media comprises 6-chloro-2-(5-chloro-2-hydroxyphenyl)-quinazolin-4(3H)-one myo-inositol-1-phosphate ammonium salt and additives for growth. The additives for growth include, but are not limited to, bovine albumin, ceftazidime, proteose peptone, tryptone, casamino acids, lab lemco powder, glucose, yeast extract, potassium phosphate, LiCl, agar, and water. This embodiment of the present disclosure can also be in powder form, containing less than 5% by weight of water. The powder form may be hydrated to yield a liquid or semi-liquid medium.

A further embodiment of the present disclosure is a method for detecting Listeria monocytogenes. L. monocytogenes are equipped with a virulence apparatus that can invade epithelial cells. This virulence apparatus secretes factors such as inositol specific phospholipase C. This embodiment of the present disclosure exploits this pathway to effectively identify and isolate L. monocytogenes. The detectable enzyme is phospholipase C, the enzyme substrate is 6-chloro-2-(5-chloro-2-hydroxyphenyl)-quinazolin-4(3H)-one myo-inositol-1-phosphate, and the fluorescent staining associated with a colony is indicative of the presence of Listeria monocytogenes.

Throughout this disclosure, unless the context dictates otherwise, the word “comprise” or variations such as “comprises” or “comprising,” is understood to mean “includes, but is not limited to” such that other elements that are not explicitly mentioned may also be included. Further, unless the context dictates otherwise, use of the term “a” may mean a singular object or element, or it may mean a plurality, or one or more of such objects or elements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A is an example of an indoxyl enzyme substrate. It depicts the general mechanism by which enzyme substrates label detectable enzymes in microbial organisms. This mechanism includes an enzyme labile group (ELG), labile bond (LB), binding motif (BM), signalogen (SG), detectable enzyme (DE) (also referred to as marker enzyme), active signalogen (aSG), and the signalophore (SP). In this example, phoshpatase, the detectable enzyme, acts upon indoxyl phosphate, the enzyme substrate, to cleave the enzyme labile group at the labile bond. This activates the signalogen which is oxidized to form blue indigo dye, the signalophore.

FIG. 1B is an example of an enzyme substrate that includes a labile spacer. This mechanism includes the same substructures as described in FIG. 1A, and in addition includes a labile spacer (LS). The labile spacer separates the signalogen and the enzyme labile group by two or more chemical bonds where the labile spacer substructure is eliminated either spontaneously following action of the detectable enzyme on the enzyme substrate at the labile bond to remove the enzyme labile group, or by other means involving chemical reagents. In this example, the chemical reagent, sodium periodate bovine serum albumin (denoted NaIO₄ BSA) will not react with the signalogen until the enzyme labile group is cleaved.

FIG. 1C is another example of an enzyme substrate that includes a labile spacer. This labile spacer features a N—C bond which allows phenolic signalogens to be linked to enzyme labile groups corresponding to amino acids or peptides.

FIG. 2 depicts an embodiment of the present disclosure in which the enzyme substrate is derived from a quinazolidone, and the signalogen is 6-chloro-2-(5-chloro-2-hydroxyphenyl)-quinazolin-4(3H)-one (ELF®).

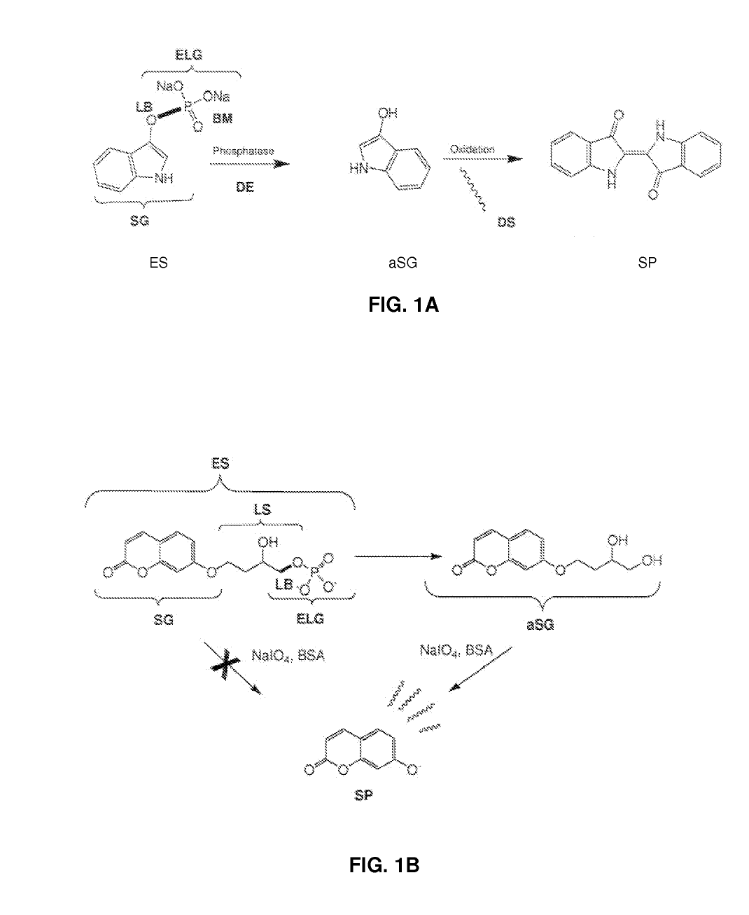

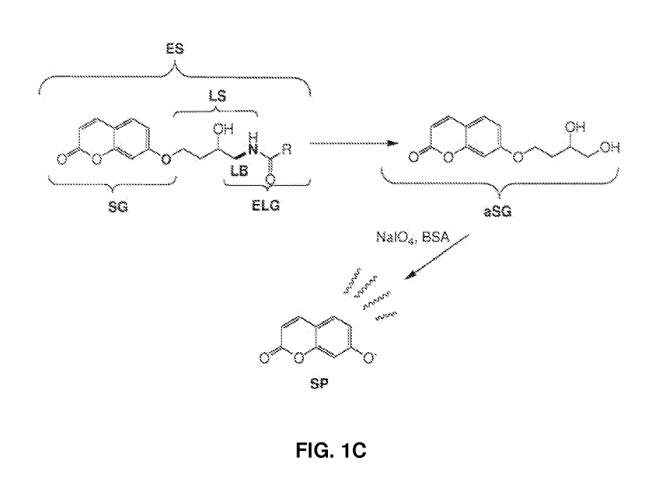

### DETAILED DESCRIPTION

### Definitions

The term “aqueous solution” as used herein refers to a solution that is predominantly water and retains the solution characteristics of water. Where the aqueous solution contains solvents in addition to water, water is typically the predominant solvent.

“Aromatic ring” refers to a hydrocarbon in which six carbon atoms are attached in a closed ring structure. In this disclosure, “aromatic ring” includes unsaturated heterocyclic ring structures.

“Binding motif” refers to a region in a protein sequence that interacts with other proteins.

“BLOCK” or “enzyme labile group” refers to a group the changes the excitation or emission properties (i.e. absorbance or fluorescence) of an attached fluorophore and is capable of being cleaved from the remainder of the substrate molecule by action of an enzyme.

“Colony” means an aggregation of cells, usually located on a growth media plate.

“Detectable enzyme” or “marker enzyme” means an enzyme of analytical interest that can be distinguished from it surrounding environment.

“Detectable label” or “Label” means a chemical used to facilitate identification and/or quantitation of a target substance. Illustrative labels include labels that can be directly observed or measured or indirectly observed or measured. Such labels include, but are not limited to, radiolabels that can be measured with radiation-counting devices; pigments, dyes or other chromogens that can be visually observed or measured with a spectrophotometer; chemiluminescent labels that can be measured by a photomultiplier-based instrument or photographic film, spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example. The label can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate or a spontaneously chemiluminescent product from a suitable precursor. The term label can also refer to a “tag” or hapten that can bind selectively to a labeled molecule such that the labeled molecule, when added subsequently, is used to generate a detectable signal. For instance, one can use biotin as a tag and then use an avidin or streptavidin conjugate of horseradish peroxidase (HRP) to bind to the tag, and then use a chromogenic substrate (e.g., tetramethylbenzidine) or a fluorogenic substrate such as Amplex Gold reagent, or a fluorescent tyramide (Molecular Probes, Inc.) to detect the presence of HRP. In a similar fashion, the tag can be a hapten or antigen (e.g., digoxigenin or an oligohistidine), and an enzymatically, fluorescently, or radioactively labeled antibody can be used to bind to the tag. Numerous labels are known by those of skill in the art and include, but are not limited to, microparticles, fluorescent dyes, haptens, enzymes and their chromogenic, fluorogenic and chemiluminescent substrates and other labels that are described in the MOLECULAR PROBES HANDBOOK OF FLUORESCENT PROBES AND RESEARCH CHEMICALS by Richard P. Haugland, 6th Ed., (1996), and its subsequent 7th edition and 8th edition updates issued on CD Rom in November 1999 and May 2001, respectively, the contents of which are incorporated by reference, and in other published sources.

“Detectable signal” means a signal that is detectable either by observation or instrumentally. Typically the detectable signal is an optical response resulting in a change in the wavelength distribution patterns or intensity of absorbance or fluorescence or a change in light scatter, fluorescence lifetime, fluorescence polarization, or a combination of the above parameters. Other detectable signals include, for example, chemiluminescence, phosphorescence, radiation from radioisotopes, attraction to a magnet and electron density.

“Enzyme” means a protein molecule produced by living organisms, or through chemical modification of a natural protein molecule, that catalyses chemical reactions of other substances without itself being destroyed or altered upon completion of the reactions. Examples of other substances, include but are not limited to chemiluminescent, chromogenic, or fluorogenic substances.

The term “fluorescent substrate” is used to describe a substrate that will produce a fluorescent product upon modification.

“Growth media plates” or “plating media” refers to a substance in which cells or micro organisms can grow. It can optionally include a supporting plate.

The term “kit” as used herein refers to a packaged set of related components, typically one or more compounds or compositions, optionally comprising buffers, separation media, standards, software and other components.

“Labile bond” means a bond that is amenable to cleavage by an enzyme.

“Signalogenic” means a molecule that can produce a detectable signal.

“Supporting plate” means a configuration that confines plating media.

“Target” means a substance of analytical interest that is analytically distinguishable from its surrounding environment.

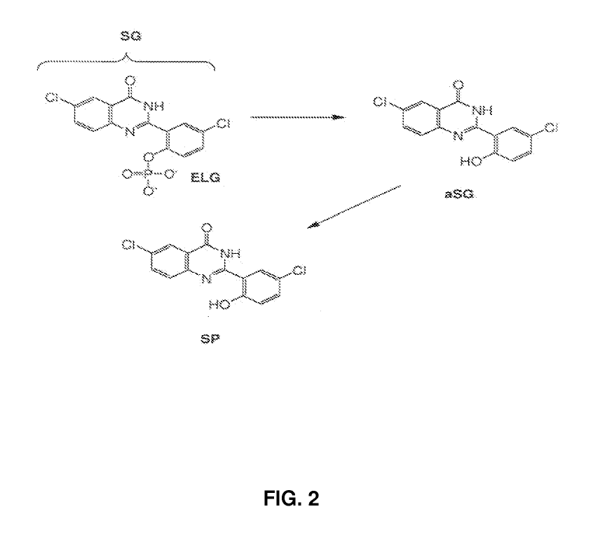

In certain embodiments of the present disclosure, enzyme substrates are derived from quinazolidones, yielding the signalphore 6-chloro-2-(5-chloro-2-hydroxyphenyl)-quinazolin-4(3H)-one (ELF®). FIG. 2 depicts a quinazolidone substrate.

U.S. Pat. Nos. 5,316,906 and 5,443,986 disclose fluorogenic enzyme substrates, however these substrates are not described for use in identification and detection of microbial organisms on plating media.

The use of 6-chloro-2-(5-chloro-2-hydroxyphenyl)-quinazolin-4(3H)-one substrates in plating media represents a significant improvement in the art by eliminating most of the shortcomings and limitations of current chromogenic and fluorogenic enzyme substrates.

For example, fluorogenic substrates capable of staining biological samples typically offer increased sensitivity and therefore higher speed and throughput of the assay. Prior to the present disclosed compositions and methods, however, there were no fluorogenic enzyme substrates available that could be incorporated into plating media and that also produced insoluble signalphores with the ability to stain microbial colonies on the plating media without killing or harming the colony forming organism in the process.

### Shortened Assay Time

While the sensitivity of any culture-based assay is limited by microbial growth since colonies must grow into a minimum size in order to become recognizable, current chromogenic plating media are limited by the performance of chromogenic indicators used and not by growth. Currently available plating media based microbial assays typically take twenty-four to forty-eight hours to produce results while colonies are often recognizable after just eight to ten hours of incubation.

The present disclosure is a significant improvement in the art because staining of colonies takes place at a much earlier stage of colony growth, usually as soon as the colonies become physically recognizable. Thus the present invention provides a significant advantage as data can typically be obtained within less than twelve hours or within half the time currently needed for a corresponding chromogenic assay.

### Insoluble Fluorescent Stain

Another major advantage of the present disclosure is that the signalogen is non-fluorescent in solution, but upon activation it spontaneously precipitates to yield a highly fluorescent insoluble stain (signalophore). In other words, the transition of active signalogen to signalophore is based on limited solubility of the active signalogen which is chemically identical to the signalophore with the difference that the former resides in a state of dissolution and the latter in an aggregated solid state. This is an improvement over the current state of the art because this disclosure combines the superior optics of fluorescence with the precise staining ability of an insoluble substrate.

Functional in Anerobic Conditions

Many microorganisms of concern belong to the groups of anerobic or microaerophilic micro-organisms which do not tolerate the concentration of oxygen needed to convert the indoxyl active signalogen into the indigo stain. Hence, novel plating media disclosed herein are potentially useful to provide plating media for anerobic or micro-aerophilic micro-organisms.

### Non-Toxic and Safe for the User

Plating media encompassing enzyme substrates that require metal ions to transform the active signalogen into the signalophore are inherently limited in their application because some microorganisms respond to the presence of transition metal ions present in the media. In fact, concentrations of metal ions are commonly used as a control parameter to select for certain organisms in microbial cultures. Even more limited are enzyme substrates that depend on the formation of azo-dyes to achieve staining Reagents commonly used in those applications are harmful to microorganisms and represent a hazard to the health and safety of laboratory staff. Thus in the context of plating media, the use or requirement of any reagents, chemical entity, or other interference, to promote the transition of an active signalogen to a signalphore represents an undesirable limitation because it can inhibit growth. Research associated with the present disclosure indicates that most microbial organisms are very tolerant towards 6-chloro-2-(5-chloro-2-hydroxyphenyl)-quinazolin-4(3H)-one substrates. For example, concentrations of 500 mg/L and higher of ELF® phosphate do not significantly inhibit the growth of S. aureus strains, which are usually very sensitive to the chemical environment and towards indoxyl substrates in particular. This embodiment of the present disclosure is a significant advantage over chromogenic plating media because S. aureus species have a low tolerance for chromogenic plating media that are primarily based on indoxyl substrates. This finding was reported in U.S. Pat. No. 6,548,268 (WO 00/53799).

### Well Suited for a Dual Reporter System

Dual reporter systems utilizing two enzyme substrates are used to differentiate microbial species or strains of the same species. For example, a dual reporter system incorporated into a plating media may include a primary enzyme substrate to indicate positive results and a secondary enzyme substrate that indicates the presence of microorganisms associated with false positive results. The second enzyme substrate must be able to stain the “false” colonies in a distinguishable manner such that ready differentiation of colonies can be achieved. Dual reporter systems based on commonly used indoxyl substrates are limited by the rather modest color range of indigo stains and the optical variation of staining caused by the level of enzyme expression and other intrinsically variable factors. As a result, indoxyl dual reporter systems often require interpretation based on inspection of colonial morphology.

The present disclosure is an improvement in the state of the art of dual reporter systems. A fluorogenic substrate can be combined with a chromogenic substrate in a dual reporter system that avoids confusion because their respective stains are fundamentally different. This design produces very clear results that can be interpreted by unskilled staff or read by automated devices, providing higher quality data and significant cost savings.

### EXAMPLES

The following examples illustrate preferred embodiments of the disclosure.

### Example 1

### Detection of Methicillin Resistant S. Aureus (MRSA)

It is well known that methicillin resistant strains of S. aureus, often referred to as MRSA, pose a significant threat to public health. For example, in 2005, MRSA caused more deaths in the United States than AIDS. Klevens et al., Invasive Methicillin-Restant Staphylococus aureus Infections in the United States, JAMA 298, 1763-71 (2007). MRSA is an especially life-threatening risk to hospital patients because MRSA often spreads through hospital facilities by infecting patients with open wounds and impaired immune systems. MRSA can be so severe that surgical amputation of infected limbs is the only means to avoid a patient's death. MRSA spreads quickly under conditions of close inter-human contact, poor health conditions, and high turnover rates—such as hospitals, prisons, and similar institutions. MRSA, however, does not originate in these types of places. Rather it is primarily brought into the environments by introduction of persons that carry the MRSA bacteria and who may or may not be infected. Thus, the only effective measure to counter the spread of MRSA involves vigorous screening of humans to be admitted to a facility at risk.

Considering the vast number of admissions of people to facilities at risk, the task of complete screening prior to admission represents a major challenge. A screening method must be fast, robust and cost efficient. Since immunological or molecular methods for detection of MRSA are expensive on a per assay basis and also require expensive laboratory equipment, cultural methods represent an attractive alternative if the assay time can be reduced.

While traditional culture methods are time consuming, chromogenic media has been used for MRSA screening even though it typically takes twenty-four hours for the assay to produce a result. Immunological or molecular methods are much faster; they typically operate on a time scale of four to eight hours. Chromogenic assays are usually preferred, however, because they are cheaper, more robust, and isolates can be produced in the same assay. Nevertheless, time remains a highly critical factor because patients must be kept in quarantine prior to admission, which is costly and cumbersome. Diekema et al., CLIN. INFECT. DIS. 44, 1101-07 (2007).

The present disclosure offers a significant improvement over chromogenic assays because ELF® based plating media can detect, isolate, and differentiate MRSA colonies in just nine to twelve hours, that is less than 50% of the time needed to perform an assay employing common chromogenic plating media.

Most chromogenic MRSA plating media use S. aureus phosphatase as the marker enzyme. Since phosphatses are wide spread the biomarker is not very specific to S. aureus. Thus, there is a need for a plating media designed so that selective growth restricts the diversity of microorganisms supported by the media.

It is known in the art that selectivity can be achieved by certain combinations of nutrients, by the addition of selective growth inhibitors such as antibiotics, or by deprivation of nutrients that will inhibit growth of undesired strains of microorganisms. Efforts to achieve complete selectivity are often hindered because strains of the target species are also inhibited. In the case of MRSA, selective agents are used to suppress the growth of many undesired species, but species such as Serratia spp., certain Bacillus spp., and Enterococci may grow nonetheless.

Additionally, these species may produce biomarker enzymes (e.g., phosphatase) that yield false positive results in the assay.

In most MRSA media two indoxyl enzyme substrates are used, one that stains colonies of phosphatse positive strains and another that stains colonies associated with beta-D-glucosidase. This type of assay is limited because S. aureus is sensitive towards indoxyl substrates and the two indoxyl substrates produce similar stains that can be difficult to visually differentiate. U.S. Pat. No. 6,548,268 describes an indoxyl dual reporter system to detect MRSA.

An MRSA media of the present disclosure includes a fluorogenic substrate, preferably ELF® phosphate for detection of S. aureus phosphate, and an indoxyl substrate such as 5-bromo-6-chloro-indolyl-beta-D-glucopyranoside for the detection of potentially interfering colonies of Bacillus spp., Enterococci or Serratia spp. The fluorescently stained MRSA can be readily detected at 360 nm UV light and the indoxyl substrate stains false positive colonies purple, thus eliminating the possibility of confusion or ambiguity.

This example illustrates preparation, enrichment, inoculation, incubation and interpretation of solid plating media according to this embodiment of the disclosure.

TABLE 1
Composition of MRSA base media for detection of acidic phosphatase

| | Enrichment | Plating |
|---|---|---|
| | broth | media |
| Base Media | g/L | g/L |
| Bacto brain heart infusion | for 1 L | — |
| Protease Peptone | — | 4 |
| Bacto Peptone | 2 | 3 |
| Tryptone | — | 3.8 |
| Yeast Extract | 2 | 3 |
| Meat Extract | — | 2 |
| NaCl | — | 5 |
| LiCl | — | 5 |
| Cycloheximid | 0.2 | 0.2 |
| Agar | — | 14 |
| Sodium pyruvate | — | 2 |
| Mannitol | — | 9 |

Ingredients listed in Table 1 were dissolved in 950 mL of distilled water, autoclaved at 121° C. for 15 minutes, allowed to cool to 50-55° C. and pH was adjusted to 7.3 with 1M NaOH.

TABLE 2
List of supplements to MRSA media

| | Enrichment | Plating |
|---|---|---|
| | broth | media |
| Supplement | g/L | g/L |
| Aztreonam | 0.0075 | 0.015 |
| Cinoxacin | 0.0025 | 0.005 |
| ELF ® Phosphate Sodium salt | — | 0.4 |
| 5-bromo-4-chloro-3-indoxyl-beta-D-galactoside | — | 0.1 |
| 5-bromo-4-chloro-3-indoxyl-beta-D-glucoside | — | 0.1 |
| Desferal | 0.2 | 0.2 |
| MgCl₂ (add separately directly to base) | — | 0.095 |
| Cefoxitin | 0.001 | 0.002 |
| Sulbactam | 0.003 | 0.006 |

The supplements (Table 2) were dissolved in a total of 50 mL of sterile distilled water, sterile micro-filtrated, and added to above media.

The plating media was poured into Petri-dishes and stored in the dark for 24 hours to allow the gel to harden. The enrichment broth was stored in sterile culture bottles.

Samples were optionally subjected to enrichment by incubation with enrichment broth prior to inoculation on plating media.

The plating media was inoculated by using a loop of liquid from sample or enrichment broth and streaking onto plating media.

The plating media was incubated at 35° C. and colony growth was monitored over time at 360 nm and under ambient light source. Data are shown in Table 3.

TABLE 3
Growth of MRSA on MRSA plating media

| Incubation Time | | |
|---|---|---|
| Hours | 360 nm | Ambient |
| 6 | Slight fluorescence | — |
| 8 | Strong fluorescence (diffuse) | Diffuse Colonies |
| 10 | Distinct fluorescent colonies | Distinct colonies |
| 12 | Same as 10 hour but more intense | Distinct colonies¹ |
| 16 | Same as 12 hour but more intense | Distinct colonies¹ |
| 24 | Same as 12 hour | Distinct colonies¹ |
| 48 | Same as 12 hour | Distinct colonies¹ |

¹False positive stains, for example certain Serratia spp. which are phosphatase positive and beta-glucosidase positive, will show light blue to blue color, compared to the white-yellow color of MRSA.

Data were collected for a wide number of MRSA strains and compared with data obtained using current commercial MRSA plating media. While selectivity of the plating media was maintained, sensitivity was consistently higher providing data on individual samples in about 50% of the time needed with current commercial products (Table 4).

The inclusion of mannitol leads to an acidification of the media during growth of S. aureus. The time for detection was decreased considerably compared to media without mannitol (Table 9, see below). Hence, using ELF®-phosphate, detection of acidic phosphatase of S. aureus is much more sensitive than detection of alkaline phosphatase.

TABLE 4
Appearance of Colony Growth After Incubation in MRSA ELF ®
Medium

| | | First appearance |
|---|---|---|
| Strain | | of colony growth |
| Number | Strain designation | (hours) |
| 11 | RKI 02/02756 | 10.5 |
| 12 | RKI 1682/06 | 9 |
| 13 | RKI 903/06 | 9 |
| 14 | RKI 1834/06 | 9 |
| 15 | RKI 1821/06 | 9 |
| 16 | RKI 1793/06 | 9 |
| 17 | RKI 1819/06 | 10.5 |
| 18 | RKI 958/06 | 9 |
| 260 | 13 ZH42 (ST217, UK)* | 18 |
| 261 | 15 ZH100 (ST225 Jap/US)* | 10.5 |
| 262 | 23 ZH39 (ST613 Zurich)* | 10.5 |
| 263 | 7 ZH70 (ST45)* | 10.5 |
| 264 | 2 ZH37 (ST45)* | 12 |
| 271 | ATCC 43300 | 14 |
| 272 | ATCC 33592 | 12 |

*Strains provided by Institute of Medical Microbiology, University of Zurich, Switzerland, Prof. Berger Bachi.

### Example 2

### Novel Plating Media for S. Aureus

Plating media for the detection, isolation and differentiation of S. aureus, a wide spread food pathogen, is readily derived from the plating media described in example 1.

The same base media can be used for MRSA and S. aureus enrichment broth and plating media. However, no supplement antibiotics are added to the base media (Table 5).

Ingredients listed in Table 1 were dissolved in 950 mL of distilled water, autoclaved at 121° C. for 15 minutes, allowed to cool to 50-55° C. and pH was adjusted to 7.3 with 1M NaOH.

TABLE 5
List of supplements to S. aureus media

| | Enrichment | Plating |
|---|---|---|
| | broth | media |
| Supplement | g/L | g/L |
| Aztreonam | 0.0075 | 0.015 |
| Cinoxacin | 0.0025 | 0.005 |
| ELF ® Phosphate Sodium salt | — | 0.4 |
| 5-bromo-4-chloro-3-indoxyl-beta-D-galactoside | — | 0.1 |
| 5-bromo-4-chloro-3-indoxyl-beta-D-glucoside | — | 0.1 |
| Desferal | 0.2 | 0.2 |
| MgCl₂ (add separately directly to base) | — | 0.095 |

The supplements (Table 5) were dissolved in a total of 50 mL of sterile distilled water, sterile micro-filtrated, and added to the above media.

The plating media was poured into Petri-dishes and stored in the dark for 24 hours to allow the gel to harden. The enrichment broth was stored in sterile culture bottles.

Typically foodstuff samples are subjected to enrichment by incubation in enrichment broth prior to inoculation on plating media.

The plating media was inoculated by using a loop of liquid from enrichment broth and streaking onto plating media.

The plating media was incubated at 35° C. and colony growth was monitored over time at 360 nm and under ambient light source. Data obtained were similar to the data for corresponding MRSA media shown in Table 3.

Data were collected for a wide number of S. aureus strains and compared with data obtained using current commercial S. aureus plating media. While selectivity of the plating media was maintained, sensitivity was consistently higher providing data on individual samples in about 50% of the time needed when using current commercial products (Table 6).

TABLE 6
Appearance of Colony Growth After Incubation in S. aureus ELF ®
Medium

| | | First appearance |
|---|---|---|
| Strain | | of colony growth |
| Number | Strain designation | (hours) |
| 1 | RKI B5 | 10.5 |
| 2 | ATCC 25923 | 9 |
| 3 | RKI H1 | 10.5 |
| 5 | RKI 1807/06 | 9 |
| 6 | RKI 1823/06 | 9 |
| 7 | RKI 1822/06 | 10.5 |
| 9 | CCM 885 | 10.5 |
| 10 | RKI 1825/06 | 9 |
| 11 | RKI 02/02756 | 9 |
| 12 | RKI 1682/06 | 9 |
| 13 | RKI 903/06 | 9 |
| 14 | RKI 1834/06 | 9 |
| 15 | RKI 1821/06 | 10.5 |
| 16 | RKI 1793/06 | 10.5 |
| 17 | RKI 1819/06 | 10.5 |
| 18 | RKI 958/06 | 9 |
| 240 | ATCC 29213 | 9 |
| 260 | 13 ZH42 (ST217, UK)* | 9 |
| 261 | 15 ZH100 (ST225 Jap/US)* | 9 |
| 262 | 23 ZH39 (ST613 Zurich)* | 9 |
| 263 | 7 ZH70 (ST45)* | 9 |
| 264 | 2 ZH37 (ST45)* | 9 |
| 271 | ATCC 43300 | 9 |
| 272 | ATCC 33592 | 9 |

*Strains provided by Institute of Medical Microbiology, University of Zurich, Switzerland, Prof. Berger Bachi.

Data were collected for various staphylococci (Table 7). S. aureus colonies started to fluoresce at about the same time when colonies became visible. Other staphylococci were differentiated by their delayed weaker fluorescence or by their beta-D-galactosidase and/or beta-D-glucosidase activity yielding blue colonies. Some species were inhibited or did not show fluorescence at all, even after prolonged incubation.

TABLE 7
Growth of various staphylococci on S. aureus media

| | First | | Colour |
|---|---|---|---|
| Species | colonies | Fluorescence | (16-20 h) |
| S. aureus | 9-12 h | 9-12 h | white-yellow |
| S. epidermidis | no growth | n.a. | n.a. |
| S. haemolyticus | 10 h | 14 h | White |
| S. lugdunensis | 12 h | 20 h | White |
| S. sciuri* | 10 h | 10 h | White |
| S. intermedius | 10 h | 10 h | light blue |
| S. hyicus | 10 h | 16 h | White |
| S. gallinarum | 10 h | 12 h | dark blue |
| S. hominis | no growth | n.a. | n.a. |
| S. carnosus | 10 h | n.a. | light blue |
| S. warneri | 10 h | 14 h | White |
| S. arlettae | 14 h | 20 h | White |
| S. saprophyticus | 10 h | 20 h | Blue |
| S. simulans | 10 h | 10 h | light blue |
| S. xylosus | 10 h | 14 h | dark blue |

*S. sciuri is a possible candidate for false positives. However it is usually not associated with humans.

### Example 3

### Tolerance of S. Aureus Towards Elf® Phosphate

Varying concentrations of ELF® phosphate in S. aureus plating media were used to determine the effect on growth and sensitivity of S. aureus to ELF® phosphate. Colony growth in the presence of ELF® phosphate was compared to colony growth in media without ELF® phosphate.

TABLE 8
Tolerance of ELF ® Phosphate by S. aureaus.

| ELF ® Phosphate | Colony | Sensitivity² |
|---|---|---|
| g/L | Growth¹ | Hours |
| 0.080 | Normal | 24-48 |
| 0.200 | Normal | 12-16 |
| 0.400 | Normal | 10-12 |
| 0.600 | Normal | 10-12 |

¹Colony growth was determined by visual inspection relative to media without ELF ® phosphate.
²Sensitivity was determined by measuring time until appearance of distinct colonies at 360 nm.

While a higher concentration of ELF® phosphate was needed in comparison to widely used indoxyl substrates (e.g., 5-bromo-6-chloroindoxyl phosphate or 5-bromo-indoxyl phosphate), the concentration of ELF® phosphate had no effect on growth. Sensitivity increased with concentration up to about 400 mg/L and then leveled off, probably due to saturation of the enzyme (Table 8).

### Example 4

### Medium for Detection of Serratia marcescens and Differentiation from Other Species Such as S. Aureus Based on the Detection of Alkaline Phosphatase as Marker Enzyme

Both Serratia marcescens and S. aureus are phosphatase positive, which can lead to false positive results when testing for the one or the other. As shown here, ELF®-phosphate is suited for easy differentiation of Serratia marcescens and S. aureus under alkaline conditions.

Ingredients listed in Table 9 were dissolved in 950 mL of distilled water, autoclaved at 121° C. for 15 minutes, allowed to cool to 50-55° C. and pH was adjusted to 7.3 with 1M NaOH.

TABLE 9
Composition of base media for detection of alkaline phosphatase

| Base | g/L |
|---|---|
| Protease Peptone | 4 |
| Bacto Peptone | 3 |
| Tryptone | 3.8 |
| Yeast Extract | 3 |
| Meat Extract | 2 |
| NaCl | 5 |
| LiCl | 5 |
| Cycloheximid | 0.2 |
| Agar | 14 |
| Sodium pyruvate | 2 |

TABLE 10
List of Supplements

| | Plating |
|---|---|
| | Media |
| Supplement | g/L |
| Polymyxin B | 0.0075 |
| Nalidixic acid | 0.010 |
| ELF ®-Phosphate | 0.4 |
| Desferal | 0.2 |
| Ferrioxamine E | 0.001 |

The supplements (Table 10) were dissolved in a total of 50 mL of sterile distilled water, sterile micro-filtrated, and added to the above media.

The plating media was poured into Petri-dishes and stored in the dark for 24 hours to allow the gel to harden.

The plating media was inoculated, incubated at 35° C. and colony growth was monitored over time (starting at 12 hours of incubation) at 360 nm and under ambient light source.

Colonies of S. aureus were clearly visible after 12 hours but started to show fluorescence only after about 14-16 hours. This is in contrast to the media detecting acid phosphatase where S. aureus fluoresce already when colonies start to be visible (see above). S. marcescens showed extremely strong fluorescence. The fluorescence of S. marcescens at 12 hours was much higher than that of S. aureus even after 20 and more hours of incubation. Therefore, under alkaline conditions ELM-phosphate allows for easy differentiation of Serratia marcescens and S. aureus.

### Example 5

### Detection of Pathogenic Listeria

While MRSA is one good example of how certain embodiments of the present disclosure provide significant value for epidemiological screening. There are many applications in other equally important fields such as food testing.

Time and cost are important factors in food testing. For example, batches of food from a production warehouse often require quality control testing. Because the food cannot be distributed until the testing is complete, the batches remain in storage until testing results are obtained. As a result, overall storage costs are directly correlated to assay time. In other words, the shorter the assay time, the lower the storage costs. Culture methods are widely used in food testing and often represent the rate limiting step on the time axis. Hence, rapid, robust, and cost-efficient methods are in high demand.

Listeria ssp. represents a major group of food borne pathogens. Not all Listeria species, however, pose a danger to humans. Infectious and pathogenic Listeria, most prominently represented by strains of L. monocytogenes, are equipped with a virulence apparatus that invades epithelial cells. The virulence apparatus produces enzyme virulence factors such as inositol specific phospholipase C. This enzyme can be detected on plating media and provides a highly effective method for identification and isolation of dangerous species. A means for detecting inositol specific phospholipase C via a chromogenic enzyme substrate was described in U.S. Pat. No. 6,051,391, which is incorporated herein in its entirety by reference.

European Patent No. 1506309 B1 describes an extension of this detection system in which a second fluorogenic substrate is added to provide an early stage indication of the presence of L. monocytogenes. The prior art is limited in that the fluorogenic substrate can not localize colonies; thus a chromogenic substrate is still required to identify and specifically stain the target colonies. There is a need, therefore, for a single step fluorogenic substrate that can specifically stain colonies that produce virulence factors such as inositol specific phospholpase C.

As shown herein, one embodiment of the present disclosure comprises a fluorogenic substrate that can rapidly stain microbial colonies of virulent Listeria spp. on plating media, thus supplanting the combination of fluorogenic and chromogenic substrates currently employed.

This embodiment of the present disclosure includes a substrate represented by a general term X—O-myo-inositol-1-phosphate where “X” corresponds to a fluorogenic substructure, “X—O” corresponds to the signalogen (SG), and myo-inositol-1-phosphate represents the enzyme labile group (ELG), also referred to as the BLOCK substructure in this disclosure.

A further embodiment of the plating media in this disclosure includes ELF® myo-inositol-1-phosphate, shown below, which replaces the currently used combination of two substrates with the added benefit of significantly accelerating the assay.

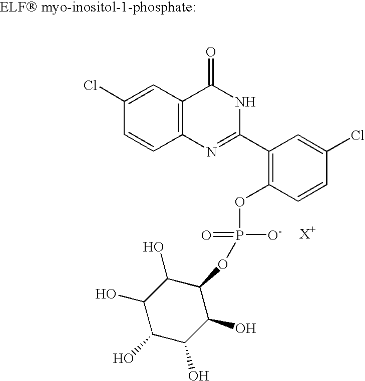

### Novel Plating Media for Listeria Monocytogenes

ELF® myo-inositol-1-phosphate ammonium salt (4-chloro-2-(6-chloro-4-oxo-3,4-dihydroquinazolin-2-yl)phenyl myo-inositol-1-phosphate) can be prepared in analogy to 4-methylumbelliferyl myo-inositol-1-phosphate disclosed in U.S. Pat. Nos. 6,051,391 and 6,068,988.

TABLE 11
Composition of L. monocytogenes base media

| | Plating media |
|---|---|
| Base Media | g/L |
| Protease Peptone | 3 |
| Tryptone | 12 |
| Casamino acids | 6 |
| Lab Lemco Powder | 5 |
| Glucose | 2.5 |
| Yeast Extract | 8 |
| Potassium phosphate (dibasic) | 4.5 |
| LiCl | 4.5 |
| Agar | 15 |

Ingredients listed in Table 11 were dissolved in 950 mL of distilled water, autoclaved at 121° C. for 15 minutes, allowed to cool to 50-55° C. and pH was adjusted to 7.3 with 1M NaOH.

TABLE 12
Supplements to L. monocytagenes media

| | Plating media |
|---|---|
| Base Media | g/L |
| Bovine albumin | 3 |
| Ceftazidime | 0.035 |
| ELF ® myo-inositol-1-phosphate | 0.8 |

The supplements (Table 12) were dissolved in 50 mL of sterile distilled water, sterile micro-filtrated, and added to above media.

The plating media was poured into Petri-dishes and stored in the dark for 24 hours to allow the gel to harden.

Samples were optionally subjected to enrichment by incubation with enrichment broth prior to inoculation on plating media.

The plating media was inoculated by using a loop of liquid from sample or enrichment broth and streaking onto plating media.

The plating media was incubated at 35° C. and colony growth was monitored over time at 360 nm and under ambient light source.

After incubation, colonies displaying the following characteristics were considered presumptive Listeria monocytogenes which was confirmed by further testing using standard Listeria monocytogenes identifying methods: fluorescent colonies, convexed, 0.2-2.5 mm in diameter depending on incubation time.

Data obtained for a number strains were collected and compared to corresponding data obtained by the use of L. monocytogenes plating media disclosed in U.S. Pat. No. 6,068,988.

While selectivity of the plating media was maintained, sensitivity was consistently higher providing data on individual samples in about 50% of the time needed when using current commercial products.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A plating media comprising an enzyme substrate represented by the general structure “BLOCK-O—X”:
a) wherein O—X is a fluorogen;
b) wherein BLOCK is an enzyme group labile to the action of a marker enzyme; and
c) wherein O—X and BLOCK are linked by an enzyme labile bond amenable to the action of a marker enzyme that cleaves BLOCK from O—X, which aggregates to yield an insoluble fluorescent precipitate.

2. A plating media comprising an enzyme substrate represented by the general structure “BLOCK-LS-O—X”:
a) wherein LS-O—X is a fluorogen;
b) wherein BLOCK is an enzyme group labile to the action of a marker enzyme; and
c) wherein LS-O—X and BLOCK are linked by an enzyme labile bond amenable to the action of a detectable enzyme resulting in the separation of BLOCK from LS-O—X and where LS-O—X represents a moiety that upon loss of LS is converted to a second moiety that aggregates to yield an insoluble fluorescent precipitate.

3. A method of detecting a microbial organism that expresses a marker enzyme comprising the steps of:
a) innoculating a plating media with a test sample, wherein the plating media comprises an enzyme substrate represented by the general structure “BLOCK-O—X”:
i) wherein O—X is a fluorogen;
ii) wherein BLOCK is an enzyme group labile to the action of a marker enzyme; and
iii) wherein O—X and BLOCK are linked by an enzyme labile bond amenable to the action of a marker enzyme that cleaves BLOCK from O—X, which aggregates to yield an insoluble fluorescent precipitate;
b) incubating the plating media for a sufficient period to obtain colonies; and
c) examining the plating media under UV light for fluorescent staining associated with an individual colony wherein the fluorescent staining is indicative of the presence of the microbial organism.

4. A method of detecting a microbial organism that expresses a marker enzyme comprising the steps of:
a) innoculating a plating media with a test sample, wherein the plating media comprises an enzyme substrate represented by the general structure “BLOCK-LS-O—X”:
i) wherein LS-O—X is a fluorogen;
ii) wherein BLOCK is an enzyme group labile to the action of a marker enzyme; and
iii) wherein LS-O—X and BLOCK are linked by an enzyme labile bond amenable to the action of a detectable enzyme resulting in the separation of BLOCK from LS-O—X and where LS-O—X represents a moiety that upon loss of LS is converted to a second moiety that aggregates to yield an insoluble fluorescent precipitate;
b) incubating the plating media for a sufficient period to obtain colonies; and
c) examining the plating media under UV light for fluorescent staining associated with an individual colony wherein the fluorescent staining is indicative of the presence of the microbial organism.

5. The composition of claim 1, wherein X has the structure: 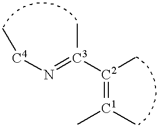
a) where carbon atoms of —C¹═C²— are further joined so as to complete a first 5- or 6-membered aromatic ring which may contain at least one of the hetero atoms N, O or S,
b) where carbon atoms of —C⁴—N═C³— are further joined so as to complete a second 5- or 6-membered aromatic ring that contains at least the nitrogen between C³ and C⁴ and may contain at least one additional hetero atom N, O or S,
c) where the first and second aromatic rings may be joined by a 5- or 6-membered bridging ring that contains at least the C² from the first aromatic ring and the C³ from the second aromatic ring, which bridging ring may be saturated or unsaturated and may contain a hetero atom N, O, or S,
d) where each of the first and second aromatic rings may be fused to at least one additional aromatic ring that may contain at least one of the hetero atoms N, O or S, and
e) where each of said aromatic rings may be further modified by substitution of any hydrogens on an aromatic carbon by substituents that are halogen, nitro, cyano, aryl, lower alkyl (1-4 carbons), perfluoroalkyl (1-4 carbons), or alkoxy (1-4 carbons), or any combination thereof; and X is covalently linked to the oxygen —O— at C¹.

6. The plating media of claim 1, wherein the detectable enzyme is a glycosidase, peptidase, esterase, carboxylesterase, lipase, cholinesterase, phosphatase, sulfatase, phospholipase A, phospholipase B, phospholipase C, dealkylase or nitroreductase.

7. The plating media of claim 6, wherein the glycosidase is alpha-amylase, alpha-D-arabinosidase, alpha-L-arabinosidase, beta-D-cellobiosidase, alpha-D-fucosidase, alpha-L-fucosidase, beta-D-fucosidase, beta-L-fucosidase, alpha-galactosaminidase, beta-galactosaminidase, alpha-galactosidase, beta-galactosidase, alpha-glucosaminidase, beta-glucosaminidase, alpha-glucosidase, beta-glucosidase, beta-glucuronidase, beta-lactosidase, alpha-maltosidase, beta-maltosidase, alpha-mannosidase, beta-mannosidase, neuraminidase, alpha-rhamnosidase, alpha-xylosidase, beta-xylosidase, alpha-L-arabinofuranosidase, beta-chitobiosidase, galactopyranoside-6-sulfatase or beta-D-ribofuranosidase.

8. The plating media of claim 6, wherein the peptidase is L-alanine aminopeptidase, aminopeptidase A, aminopeptidase B, aminopeptidase M, dipeptidyl-aminopeptidase I, dipeptidyl-aminopeptidase II, dipeptidyl-aminopeptidase III, dipeptidyl-aminopeptidase IV, gamma-glutamyl transferase, hippurase, L-proline aminopeptidase, proline arylamidase, prolyl endopeptidase, proglutamyl peptidase I or ureidase.

9. The plating media of claim 6, wherein the phosphatase is alkaline phosphatase, acidic phosphatase, phosphodiesterase, endo pyrophosphatase, exo pyrophosphatase, DNAse or ATPase.

10. The plating media of claim 6, wherein the sulfatase is arylsulfatase or glycosulfatase.

11. The plating media of claim 6, wherein the phospholipase C is inositol specific phospholipase C or choline specific phospholipase C.

12. The plating media of claim 1, wherein BLOCK is a blocking group that is a monovalent moiety derived by removal of a hydroxyl group from phosphate or sulfate or a biologically compatible salt thereof; or a monovalent moiety derived by removal of a hydroxyl group from a carboxy group of an aliphatic, aromatic or amino acid or of a peptide; or a monovalent moiety derived by removal of an anometic hydroxyl group from a mono- or polysaccharide; and is capable of being cleaved from the remainder of the substrate by action of a specific enzyme.

13. The plating media of claim 1, wherein the media is in powder form.

14. The plating media of claim 1, further comprising a supporting plate.

15. The plating media of claim 1, wherein X is 6-chloro-2-(5-chloro-2-hydroxyphenyl)-quinazolin-4(3H)-one (referred to as ELF®).

16. The method of claim 3, wherein the microbial organism is a methicillin resistant strain of S. aureus (MRSA), the detectable enzyme is S. aureus phosphatase, the enzyme substrate is 6-chloro-2-(5-chloro-2-hydroxyphenyl)-quinazolin-4(3H)-one phosphate, and the fluorescent staining observed under 360 nm ultraviolet light is indicative of the presence of MRSA.

17. The method of claim 16 further comprising the step of detecting colonies of Bacillus spp., Enterococci, or Serratia spp. by the addition of an indoxyl substrate to the plating media wherein a purple colony is indicative of colonies of Bacillus spp., Enterococci, or Serratia spp.

18. A plating media for the detection of MRSA comprising 6-chloro-2-(5-chloro-2-hydroxyphenyl)-quinazolin-4(3H)-one phosphate sodium salt and additives for growth.

19. A plating media for the detection of MRSA comprising 6-chloro-2-(5-chloro-2-hydroxyphenyl)-quinazolin-4(3H)-one phosphate sodium salt, 5-bromo-4-chloro-3-indoxyl-β-D-glucopyranoside, and additives for growth.

20. The plating media of claim 18 wherein the additives for growth comprise cefoxitine, sulbactam, plolymyxin B, desferrioxamine B, ferrioxamin E, proteose peptone, bacto peptone, tryptone, yeast extract, meat extract, NaCl, LiCl, cycloheximid, agar, and water.

21. The plating media of claim 18 in powder form.

22. The method of claim 3, wherein the microbial organism is S. aureus, the detectable enzyme is S. aureus phosphatase, the enzyme substrate is 6-chloro-2-(5-chloro-2-hydroxyphenyl)-quinazolin-4(3H)-one phosphate, and the fluorescent staining observed under 360 nm ultraviolet light is indicative of the presence of S. aureus.

23. A plating media for the detection of S. aureus comprising 6-chloro-2-(5-chloro-2-hydroxyphenyl)-quinazolin-4(3H)-one phosphate sodium salt, 5-bromo-4-chloro-3-indoxyl-β-D-glucopyranoside, desferrioxamine B, and Ferrioxamin E.

24. A plating media for the detection of Listeria monocytogenes comprising 6-chloro-2-(5-chloro-2-hydroxyphenyl)-quinazolin-4(3H)-one myo-inositol-1-phosphate ammonium salt and additives for growth.

25. The plating media of claim 24, wherein the additives for growth comprise bovine albumin, ceftazidime, proteose peptone, tryptone, casamino acids, lab lemco powder, glucose, yeast extract, potassium phosphate, LiCl, agar, and water.

26. The plating media of claim 24 in powder form.

27. The method of claim 3, wherein the microbial organism is Listeria monocytogenes, the detectable enzyme is phospholipase C, the enzyme substrate is 6-chloro-2-(5-chloro-2-hydroxyphenyl)-quinazolin-4(3H)-one myo-inositol-1-phosphate, and the fluorescent staining observed under 360 nm ultraviolet light is indicative of the presence of Listeria monocytogenes.

28. The plating media of claim 27 in powder form.

29. A package containing a plurality of growth media plates comprising 6-chloro-2-(5-chloro-2-hydroxyphenyl)-quinazolin-4(3H)-one myo-inositol-1-phosphate ammonium salt and additives for growth.

30. A package containing a plurality of growth media plates comprising 6-chloro-2-(5-chloro-2-hydroxyphenyl)-quinazolin-4(3H)-one phosphate sodium salt, 5-bromo-4-chloro-3-indoxyl-β-D-glucopyranoside, desferrioxamine B, and ferrioxamin E.

31. A package containing a plurality of growth media plates comprising 6-chloro-2-(5-chloro-2-hydroxyphenyl)-quinazolin-4(3H)-one phosphate sodium salt, 5-bromo-4-chloro-3-indoxyl-β-D-glucopyranoside, and additives for growth.
